(19) United States
(12) Patent Application Publication
(10) Pub. No.: US 2007/0270678 A1
(43) Pub. Date: Nov. 22, 2007

(54) Wireless Electrode for Biopotential Measurement

(76) Inventors: Kalford C. Fadem, Louisville, KY (US); Benjamin A. Schnitz, Brentwood, TN (US)

Correspondence Address: FROST BROWN TODD, LLC, 2200 PNC CENTER, 201 E. FIFTH STREET, CINCINNATI, OH, 45202, US

(21) Appl. No.: 11/570,626

(22) PCT Filed: Jun. 16, 2005
(86) PCT No.: PCT/US05/21257

§ 371 Date: Jul. 18, 2007

Related U.S. Application Data
(60) Provisional application No. 60/580,772, filed on Jun. 18, 2004.
(60) Provisional application No. 60/580,776, filed on Jun. 18, 2004.

(51) Int. Cl. A61B 5/04

(52) U.S. Cl. 600/372

(57) ABSTRACT

A wireless biopotential monitoring system composed of a wireless electrode module which can be attached to a disposable electrode strip. Such device can be conveniently affixed to a patient's skin and will transmit the physiological signals to a remote receiver where the signals can be monitored by a clinician.

### CROSS REFERENCE TO RELATED APPLICATIONS

The present application claims the benefit of U.S. patent application Ser. No. 60/580,776 “DEVICE AND METHOD FOR TRANSMITTING PHYSIOLOGIC DATA” and 60/580,772 “WIRELESS ELECTRODE FOR BIOPOTENTIAL MEASUREMENT” both to Fadem et al. and filed on 18 Jun. 2004, the disclosure of both of which are incorporated by reference in its entirety.

### FIELD OF THE INVENTION

The present invention relates generally to a method and apparatus for capturing biopotential voltage signals such as electroencephalograms (EEG's), electrocardiograms (ECG's) or electromyograms (EMG's). More particularly, the present invention provides a method and describes a battery powered device which uses a digital amplification circuit attached to a disposable adhesive electrode strip to capture voltage potentials from the surface of the skin and a digital wireless transceiver tightly integrated with respect to the amplification circuit to send the voltage potential signals to a remote receiver for datalogging.

### BACKGROUND OF THE INVENTION

The measurement of voltage potentials from the surface of the skin are commonly used to detect a variety of physiological conditions. Voltage potentials generated by the beating heart called ECG's are used to evaluate the performance and condition of the heart and may be indicative of many types of heart disease. EMG's are often detected from electrodes affixed to the skin near muscles to evaluate a subject's neuromuscular activity and may be used to identify muscular dystrophy, peripheral nerve damage or other diseases. EEG's are voltage potentials generated by activity within the brain. EEG's are detected by placing electrodes on the scalp and are often used to detect neurological conditions such as schizophrenia, auditory neuropathy, or the effects of anesthesia.

These voltage potentials are measured by affixing a plurality of conductive electrodes, at least one of which, the reference electrode, should be placed at a site of minimal electrical activity, and measuring the voltage differential between the reference electrode and the other, signal, electrodes. The electrodes are commonly made from a conductive material such as silver/silverchloride (Ag/AgCl) or gold (Au) and are often wetted with a conduction enhancing solution such as saline or a conductive gel.

The voltage differential between the reference electrode and the signal electrodes is extremely small, on the order of millivolts (10⁻³ volt) or microvolts (10⁻⁶ volt). To detect the small physiological signal in the presence of background electrical noises requires amplification and filtering. The amplification and filtering is usually accomplished via an amplifier box connected to the electrodes with long wires.

For many applications of biopotential measurement, the long electrode wires present a number of problems both in terms of the utility of the system and the accuracy of the measurements.

Often it is desirable to monitor EEG's, ECG's, or EMG's in a clinical environment such as an ambulance, an emergency room, an operating room, or a recovery room. These environments are often cluttered with tubes and wires from the various life support or physiological monitoring equipment attached to the patient. Reducing the number of physical connections from the equipment to the patient, thereby decreasing the tangle of tubes and wires, would permit care givers to work more efficiently around the patient.

Affixing a multitude of individual electrodes to the patient's skin and attaching the other ends to an equipment box also requires a significant amount of time. Depending on the type of electrodes used, the location on the body where the electrodes are to be attached, and the type of biopotential signals to be measured, many system parameters have to be set. These parameters may include settings for gain, filter characteristics, and sampling frequency. This extended set-up time, up to thirty minutes for many EEG or ECG systems, may be significant for many patients in need of critical attention.

Not only are the long attachment wires burdensome themselves, the wires also tend to limit the accuracy of the electrophysiological signals being detected. This is for a number of reasons. First, the wires act as an antenna which will pick up stray background electrical noise. This background noise could come from other powered equipment or from electrosurgical devices used to cauterize wounds. Electrical filters in the amplifier box are used to limit the degradation caused by background noise but in doing so, also mask or modify a certain amount of the signal. The second reason that long wires limit the accuracy of the detected signals is that because the signals are very small, on the order of millivolts (10⁻³ volt) or microvolts (10⁻⁶ volt), there is a certain amount of signal loss due to the impedance of the wire. Finally, as the physicians and nurses work around the patient, the wires are often disturbed. Disturbing the wires can create noise and cause signal degradation.

Aspect Medical markets the BIS system, described in U.S. Pat. No. 6,298,255, for measuring EEG's to evaluate sedation levels during surgery. While the BIS system includes electrode contacts, like those described in U.S. Pat. No. 5,305,746, and an identifier memory chip affixed to an adhesive strip, this strip must be plugged into an interface box which is in turn plugged into a monitor. While this system and the device described in U.S. Pat. No. 6,654,626 do shorten setup time, these systems still require a cable between the electrode strip and the monitor. This wire can get in the way of the care givers and, if disturbed, could cause the electrode strip to become detached. The long electrode wire can also cause signal noise and degradation.

Physiometrix likewise markets the PSA 4000 system. This system also includes an adhesive electrode strip connected by a long wire to an interface box then into a monitor. This system suffers from many of the same shortcomings previously mentioned.

BioSemi markets a preamplified electrode for biopotential measurements. With this system, BioSemi has developed an electrode contact with integrated amplifiers. This system has the advantage of amplifying the signal close to the contact point. The signals are then sent along a wire to a junction box where the signal is amplified again and then converted to a digital signal. While this system amplifies a cleaner signal, the long wires between the electrode and the junction box are still problematic. This system also requires an additional amplification step before the signal is digitized so that any noise picked up from the long wire will be included in the digitized signal. The BioSemi system requires an additional wire attached to a separate reference electrode.

Thought Technology LTD markets a variety of biopotential electrodes:

MyoScan-Pro, MyoScan, and EEG-Z. These are preamplified electrodes which can be attached to an integrated electrode strip. This system, like the BioSemi system, amplifies the signal close to the electrode contact but uses long electrode wires to send the signal to an interface box for analog to digital conversion.

Cleveland Medical Devices markets the Crystal Monitor and the BioRadio Jr. The Crystal Monitor is a wireless interface/junction box which accepts standard, non-amplified, wired electrodes. This system, described in U.S. Pat. No. 5,755,230 eliminates the need for the wires between the junction box and the monitor but still uses discrete wired electrodes affixed to the skin. This system does not amplify the signal close to the skin contact point. Instead, standard wired electrodes are affixed to the skin and are attached by long electrode wires to the wireless junction box. This does not eliminate the problems associated with the clutter of wires and signal degradation can occur because of the long electrode wires.

The BioRadio Jr. does include a signal amplifier, an analog to digital converter, and a radio transmitter, and is battery powered, but this system does not utilize a preformed, adhesive electrode strip. The device, as described in their marketing literature, does not include a practical packaging arrangement. There is also no discussion of a method to automatically identify the specific biopotential measurement taken and therefore there is no method to preset the signal gain, filtering, or data capture or transmit rate.

U.S. Pat. No. 6,577,893 describes a wireless sensor device which can include sensors for biopotential measurement. This device is deficient for biopotential measurements for several reasons. First, the sensors are packaged close together and do not provide enough separation between the signal and reference electrodes to get an accurate measurement of voltage potential. Next, the device does not include a disposable contact to ensure sterility. The device also does not include an identification chip to facilitate automatic system configuration.

U.S. Pat. No. 6,611,705 describes a system and method to measure the biopotential signals related to an electrocardiograph (ECG). This system is primarily a replacement for the wires between the electrode junction box and the monitor commonly used in existing ECG systems. While this system does eliminate this wired connection, other issues of usability are not addressed.

Consequently, a significant need exists for an improved device for obtaining and wirelessly transmitting biopotential data received from a patient.

### BRIEF SUMMARY OF THE INVENTION

The invention overcomes the above-noted and other deficiencies of the prior art by providing a wireless biopotential measuring device with improved signal detection that is simple to set up and use in a clinical environment.

A device is described which includes a means to automatically configure biopotential measurement parameters, a means to detect biopotential signals from the surface of skin, a means to amplify the biopotential signals, and a means to wirelessly transmit the signals to a remote monitor.

A method of transmitting biopotential signals from a patient, comprising the steps: sampling voltage differentials between a reference electrode and a signal electrode; amplifying the voltage differentials; converting the voltage differentials to a digital format; storing a plurality of digital samples in a memory device; and transmitting the stored samples via a wireless transmitter while continuing to sample.

A biopotential measurement device affixable to skin of a patient, comprising: an adhesive substrate; a disposable electrode strip disposed on the adhesive substrate to position a pair of electrode contacts; voltage potential detection circuitry responsive to a biopotential signal across the pair of electrode contacts; processing circuitry operatively configured to signal amplify and digital convert the sensed biopotential signal; and wireless data transmission circuitry operatively configured to transmit the amplified, digitized biopotential signal.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a perspective view of a wireless biopotential measurement device.

FIG. 2 is an exploded view of the device in FIG. 1.

FIG. 3 is a close-up view of an unfolded flex circuit which is used in the device in FIG. 1.

FIG. 4 is a perspective view of an alternative embodiment of the electrode strip used in the device in FIG. 1.

FIG. 5 is a perspective view of a battery charging and wireless receiver.

FIG. 6 is a cross-section view from FIG. 1 of an electrode pad used in the device in FIG. 1.

FIG. 7 is a cross-section view from FIG. 1 of an alternative embodiment of an electrode pad used in the device in FIG. 1.

FIG. 8 is a functional block diagram of the circuit used in the device in FIG. 1.

FIG. 9 is a functional block diagram showing the signal communication path.

FIG. 10 is a functional block diagram of an alternative signal communication path.

### DETAILED DESCRIPTION OF THE INVENTION

A sealed electronics module is described which encloses a flexible printed circuit with various integrated circuit devices attached. These integrated circuits include amplifiers, analog to digital converters, a microcontroller, random access memory, and a digital radio. Also included in the module are a battery and an antenna integrated onto the flexible circuit.

The invention also includes a flexible electrode strip with at least one electrode contact affixed to each end. A memory chip containing a digital identifier is affixed to the electrode strip. Contact plugs are affixed to the electrode strip and are electrically connected to electrode pads and to the identifier memory chip.

The electrode strip has an adhesive backing so that it can be adhesively affixed to a location on a subject's skin, such as the forehead. The electrode contacts may be impregnated with an electrolytic substance to enhance the skin conductance. Once the electronics module is attached to the electrode strip by inserting the electrode strip plugs into the mating sockets on the electronics module, the device becomes electrically energized. The electronics module reads the identification data from the contact strip and configures itself for the appropriate gain, data capture rate, and wireless transmission rate.

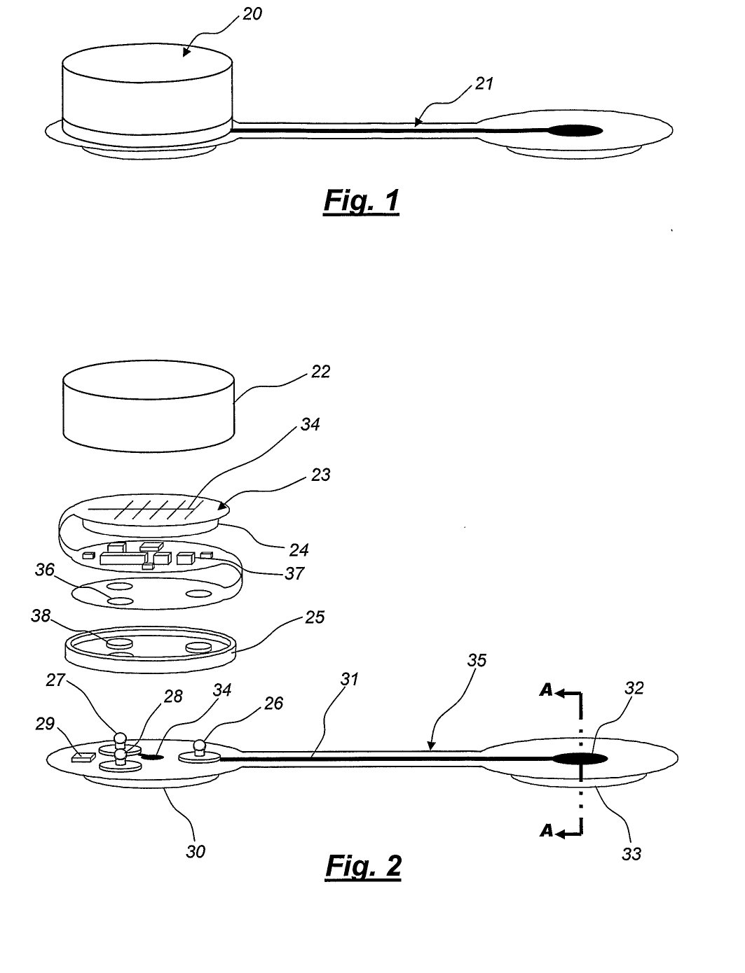

Turning to the Drawings, in FIG. 1, the wireless electrode module 20 is a sealed package which can be attached to an electrode strip 21.

In FIG. 2, electrode module cover 22 has been separated from electrode module base 25 to reveal the flexible circuit assembly 23. The flexible circuit assembly 23 has electrical contacts 36 which are electrically connected to the integrated circuit components 37. An antenna 34 and a battery 24 are also electrically connected to the integrated circuit components 37. The flexible circuit assembly 23 is assembled to the electrode module base 25 with the use of solder or conductive glue between electrical contacts 36 and electrode receptacles 38 which are permanently affixed to electrode module base 25. The wireless electrode module 20 is connected to the electrode strip 35 by inserting contact conductor plugs 26-28 into electrode receptacles 38 which are electrically connected to reference contact 32, signal contact 34, and the identification memory chip 29. The identification memory chip 29 stores the parameters for the specific desired biopotential measurement. These parameters may include: signal gain, filter settings, sampling rate, and transmission rate. The signal conductive adhesive pad 30 is affixed to the skin of a test subject in close proximity to the location desired for the biopotential measurement. The reference conductive adhesive pad 33 is affixed to the skin at a location of minimal electrophysiological activity such as the forehead.

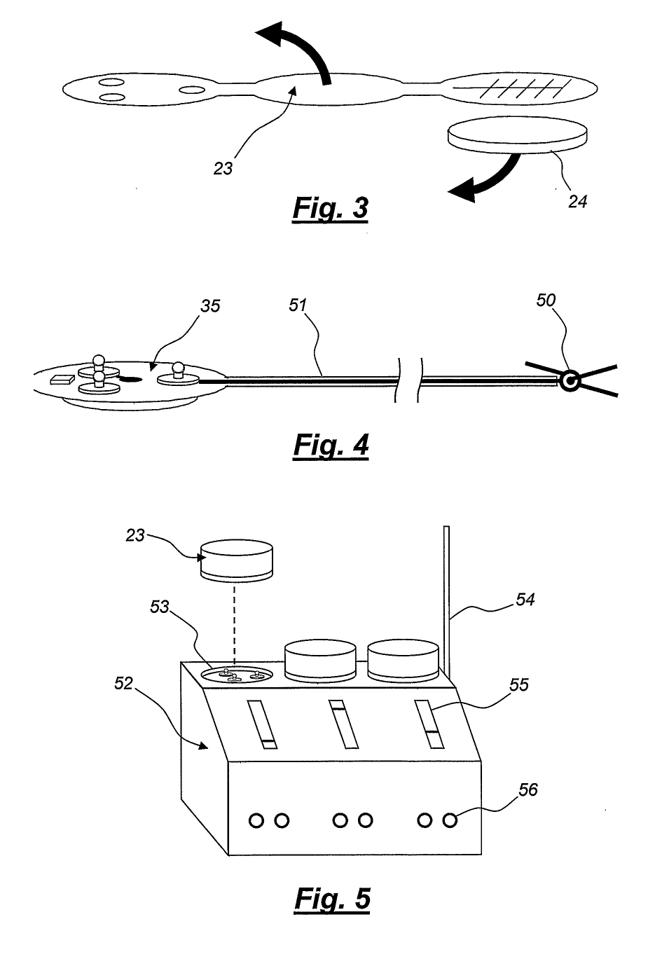

FIG. 3 shows flexible circuit assembly 23 in its unfolded configuration. Battery 24 is shown before being attached to flexible circuit assembly 23.

FIG. 4 shows an alternative configuration of electrode strip 35 where the reference conductive adhesive pad 33 has been replaced with a reference conductive clip 50 attached to a tab 51 on electrode strip 35. In this configuration, the signal conductive adhesive pad 30 is affixed to the skin of a test subject in close proximity to the location desired for the biopotential measurement. The reference conductor clip 50 is clipped to the skin at a location of minimal electrophysiological activity such as the ear lobe.

FIG. 5 shows the charging stand and wireless receiver 52. The electrode module 23 is placed in the charging sockets 53 when needing to be recharged. The charge state of the electrode module 23 is shown on charge display 55. When the electrode module 23 is in use, the biopotential signals transmitted from the electrode module 23 are received through the receiving antenna 54 and converted and sent to a patient monitor 71 through the signal output ports 56.

FIG. 6 shows a section through the electrode pad 32 from FIG. 2. The contact pad 33 may be impregnated with a conduction enhancing substance such as saline. Adhesive flanges 41 surrounding the contact pad 33 on the electrode strip 35 may be coated with an adhesive 40 to facilitate the contact pad 33 maintaining constant pressure on the skin.

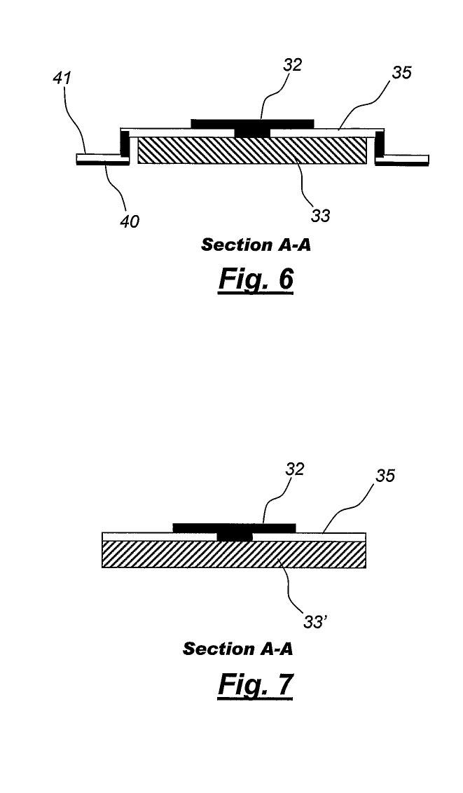

FIG. 7 shows a section through the electrode pad 32 from FIG. 2 in an alternative configuration. In this configuration, the electrode pad 33′ is coated with an adhesive which also enhances the skin conduction.

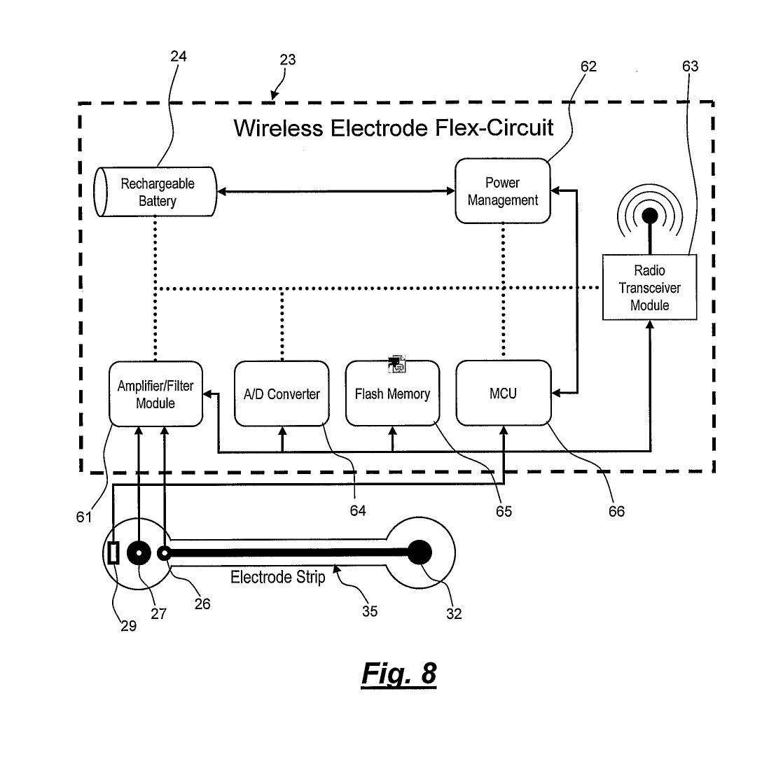

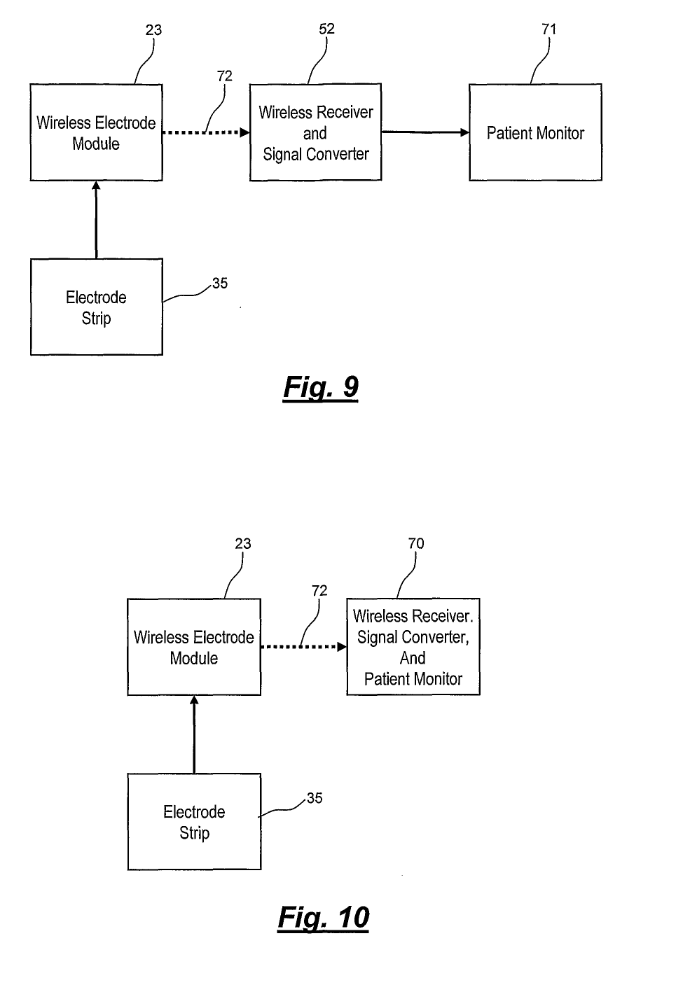

FIG. 8 shows a functional block diagram of electrode module 23 and electrode strip 35. Upon mating the electrode module 23 with the electrode strip 24, the microcontroller unit detects the electrical connection with identification memory chip 29 and energizes the combined system. Reference contact 32 and signal contact 27 become electrically connected to amplifier/filter module 61 which is connected to A/D converter 64, flash memory 65, microcontroller unit 66, and radio transceiver module 63. Identification memory chip 29 affixed to electrode strip 35 is electrically connected to microcontroller unit 66. Rechargeable battery 24 is connected to power management unit 62, amplifier/filter module 61, A/D converter 64, flash memory 65, microcontroller unit 66, and radio transceiver module 63. Additional information stored on identification memory chip 29 is read by microcontroller unit 66 which sets parameters for signal gain, filter settings, sampling rate, and transmission rate thus completing system initialization. Microcontroller unit 29 then activates the electrode by sending a Chip Select command and then clocks the data out. The amplified voltage potentials are then either transmitted wirelessly via radio transceiver module 63 or are temporarily stored in flash memory 65 and then transmitted in short bursts to increase battery life.

FIG. 10 shows a functional block diagram of the communication path of the detected biopotential signals using the described device. Electrode module 23 is electrically connected to electrode strip 35 which is placed on the skin. The voltage differentials are detected, amplified, and digitized in electrode module 23. The digital signal is then transmitted wirelessly 72 to wireless receiver 52. The signal is then converted to a signal which can be read by existing systems and sent via wire to an existing patient monitor 71.

FIG. 11 shows a functional block diagram of an alternative configuration for the communication path. Electrode module 23 is electrically connected to electrode strip 35 which is placed on the skin. The voltage differentials are detected, amplified, and digitized in electrode module 23. The digital signal is then transmitted wirelessly 72 to the combination wireless receiver and patient monitor 70.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

For example, U.S. Patent No. entitled “ACTIVE, MULTIPLEXED DIGITAL NEURO ELECTRODES FOR EEG, ECG, EMG APPLICATIONS”, Ser. No. 60/557,230, filed on 29 Mar. 2004, subsequently filed as U.S. patent application Ser. No. 11/092,395 and WO 05/010515 both on 29 Mar. 2005, the disclosures of which are hereby incorporated by reference in their entirety, all describe a novel amplified digital electrode for biopotential measurements. The disclosed electrode detects, amplifies, and digitizes the voltage potential at the point of skin contact, thereby minimizing signal noise and degradation.

What is claimed is:

1. A biopotential measurement device, comprising:
a voltage potential detection means;
a signal amplification and digital conversion means;
a wireless data transmission means; and
a disposable electrode strip.

2. The device of claim 1, further comprising an automatic configuration means to preconfigure the device with the appropriate settings relating to biopotential measurement type, signal gain, filter configuration, sampling rate, or transmission rate.

3. The device of claim 1, further comprising a rechargeable battery integrated into the electronics module.

4. The device of claim 3, further comprising a battery recharging stand.

5. The screening device of claim 4, further comprising a battery charge display means integrated into the recharging stand.

6. The screening device of claim 4, further comprising a wireless receiver means used to receive data from the wireless data transmission means.

7. The screening device of claim 1, further comprising a wireless receiver means used to receive data from the wireless data transmission means.

8. The screening device of claim 1, further comprising a memory chip identification means affixed to the disposable electrode strip used to preconfigure the measurement settings.

9. The screening device of claim 1, further comprising a battery integrated into the disposable electrode strip.

10. The screening device of claim 8, further comprising a battery that is only activated upon contact with air.

11. The device of claim 1, further comprising a means to energize the device upon connection to the electrode strip.

12. The device of claim 1, further comprising a conductive clip to be used as a reference electrode.

13. The device of claim 6 and claim 7, further comprising a signal modification means to convert the received signal such that it can be used by an existing EEG, ECG, or EMG monitor.

14. The device of claim 6 and claim 7, further comprising a monitor to display the measured biopotential signals.

15. A method of transmitting biopotential signals from a patient, comprising the steps:
sampling voltage differentials between a reference electrode and a signal electrode;
amplifying the voltage differentials;
converting the voltage differentials to a digital format;
storing a plurality of digital samples in a memory device; and
transmitting the stored samples via a wireless transmitter while continuing to sample.

16. The method of claim 15, further comprising:
accessing a subject identifier associated with the patient; and
transmitting the subject identifier with the stored samples.

17. The method of claim 15, wherein transmitting the stored samples further comprises:
disabling transmission;
receiving a remotely sent coded signal; and
transmitting the plurality of digital samples in response to verifying authenticity of the remotely sent coded signal.

18. A biopotential measurement device affixable to skin of a patient, comprising:
an adhesive substrate;
a disposable electrode strip disposed on the adhesive substrate to position a pair of electrode contacts;
voltage potential detection circuitry responsive to a biopotential signal across the pair of electrode contacts;
processing circuitry operatively configured to signal amplify and digital convert the sensed biopotential signal; and
wireless data transmission circuitry operatively configured to transmit the amplified, digitized biopotential signal.

19. The biopotential measurement device of claim 18, wherein the processing circuitry further comprises a stored identifier associated with the patient, the wireless data transmission circuitry further operatively configured to transmit the stored identifier with the amplified, digitized biopotential signal.

20. The biopotential measurement device of claim 18, wherein the wireless data transmission circuitry is further operatively configured to receive a remotely sent enablement signal, the processing circuitry further operatively configured to verify authenticity of the remotely sent enablement signal and in response thereto to enable transmission of the amplified, digitized biopotential signal.
